(19) United States
(12) Patent Application Publication
(10) Pub. No.: US 2009/0280146 A1
(43) Pub. Date: Nov. 12, 2009

(54) CONTROL OF INTESTINAL INFLAMMATORY SYNDROMES WITH A PREPARATION OF KILLED OR NON INFECTIOUS BACTERIA

(75) Inventors: Gilles Marchal, Ivry Sur Seine (FR); Micheline Lagranderie, Neuilly (FR); Mohammad Abolhassani, Paris (FR); Herve Bercovier, Jerusalem (IL)

Correspondence Address: OBLON, SPIVAK, MCCLELLAND MAIER & NEUSTADT, L.L.P., 1940 DUKE STREET, ALEXANDRIA, VA, 22314, US

(73) Assignees: INSTITUT PASTEUR, Paris (FR); YISSUM RESEARCH DEVELOPMENT COMPANY, Jerusalem (IL)

(21) Appl. No.: 12/158,827

(22) PCT Filed: Dec. 21, 2006
(86) PCT No.: PCT/IB06/04133

§ 371 Date: Oct. 21, 2008

(30) Foreign Application Priority Data
Dec. 21, 2005 (CA) 2531261

(51) Int. Cl. A61K 39/04; A61K 39/02; A61K 35/74; C12N 5/10; A61P 37/02; A61P 1/04; A61P 1/00

(52) U.S. Cl. 424/248.1; 424/234.1; 424/93.4; 435/252.1; 435/253.1

(57) ABSTRACT

This invention relates to the use of a preparation of killed or non infectious Gram positive bacteria such as Gram positive facultative intracellular bacteria, for example mycobacteria, for the treatment of intestinal inflammatory syndromes such as Crohn's disease or ulcerative colitis.

### FIELD OF THE INVENTION

This invention relates to the use of a preparation of killed or non infectious Gram positive bacteria such as Gram positive facultative intracellular bacteria, for example mycobacteria, for the treatment of intestinal inflammatory syndromes.

### DESCRIPTION OF THE PRIOR ART

With an increase in incidence, Crohn's disease is a disease found in children, adolescents and adults which can be serious, and requires specialized long term attention. Ulcerative colitis is also an Inflammatory Bowel Disease (IBD) which is disabling, of unknown etiology, and which affects young subjects. The cumulative prevalence of these two diseases is 10 to 200 per 100 000 inhabitants depending on the country. Recent dietary modifications and bacterial flora imbalances have been implicated in the origin of these afflictions, without precise results being reported. For Crohn's disease, bacteria of the genus Mycobacterium avium sub. sp. paratuberculosis have been proposed to be at the origin of the disease in part because of its analogy with Johne's disease, a disease affecting cattle which appears a few years after the initial infection. More recently, E. coli adhesins, flagellin or fimbriae, have been implicated in the pathology of the disease.

Idiopathic inflammatory bowel disease (IBD) includes a collection of disorders of the gastrointestinal tract of unknown aetiology, characterized by intestinal inflammation and a chronic relapsing course associated with local and systemic complications.

The aetiology of IBD remains unclear, but it is well established that the lesions and symptoms are associated with over-production of pro-inflammatory cytokines. IBD comprises two entities, ulcerative colitis (UC) and Crohn's disease (CD) and an intermediate variant of these diseases, indeterminate colitis which shows overlapping features of the two major forms.

The immune response is implicated in these inflammatory bowel diseases. The clinical table, by successive advances, and with extended periods of remission, has evoked the participation of autoimmune mechanisms; however, are these causes or consequences of the diseases? The active treatments brought about particularly by anti-TNF-α, underline that immunological mechanisms in the broad sense are implicated in these diseases, once again without providing any precise indication of the initial mechanism.

The different murine experimental models partially reproduce human diseases. It has been shown that flora participates to the disease. Mice maintained in a germ free breeding environment do not develop inflammatory syndrome by contrast to the mice in which the flora is conventional. Some of these inflammatory bowel disease (IBD) models in the rat or in the mouse involve a local sensitization by phenolic derivatives (TNBS) deposited locally in the colon or by the ingestion of dextran sodium sulphate (DSS) given in the drinking water for a few days. Regulatory T lymphocytes have been described to play a role in IBD models.

Intestinal inflammatory syndromes are chronic pathologies which are disabling and which may be mortal since they bring about intestinal stenoses and repetition of necessary surgical interventions. The medical treatment for these inflammatory syndromes is essentially based on the use of powerful anti-inflammatory agents, corticoids, antimitotics, and more recently anti-TNF agents. Any treatment bringing about a decrease in the doses of anti-inflammatory agents to be administered or that may be substituted to these anti-inflammatory agents are important.

In the course of analyzing mechanisms that may control experimental asthma in mice, it was discovered that CD4+ CD25+ regulatory cells are produced. The IL-10 that is secreted in turn insures an important part of the anti-inflammatory activity. The important decrease in the number of inflammatory cells present after the administration of an allergen in the lungs of the animals that were treated with Mycobacteria bovis BCG killed by Extended Freeze Drying (EFD) led the inventors to determine the eventual activity of EFD in other syndromes where immunoallergic or autoimmune phenomena have been described. In models reproducing rheumatoid arthritis, the participation of CD4+ T lymphocytes was also reported, the presence or transfers of CD4+ CD25+ T lymphocytes can stop the disease.

### SUMMARY OF THE INVENTION

An object of the present invention is to provide a treatment for intestinal inflammatory syndromes such as inflammatory bowel disease (IBD).

Another object of the present invention is to provide a treatment for IBD which does not require or decrease the dose of anti-inflammatory agents required. Accordingly, the present invention provides the use of a Gram positive bacteria preparation for the prevention and treatment of intestinal inflammatory syndromes, the preparation being characterized in that the Gram positive bacteria are killed or non infectious, and contains more than 50%, and preferentially more than 90%, of the bacterial protein components which are in a native structure.

The present invention further provides a method for preventing or treating a disease caused by a Th1/Th2 imbalance, the method comprising the steps of:
a. providing a killed or non infectious Gram positive bacteria preparation or a portion thereof retaining the capacity to inhibit intestinal inflammatory syndromes, and
b. administering an effective amount of the killed Gram positive bacteria preparation or the portion thereof to a patient affected by the disease.

The present invention still provides a method for preventing or treating an intestinal inflammatory syndrome, the method comprising the step of administering to a patient an effective amount of a Gram positive bacteria preparation, thereby stimulating the production of leukocyte regulatory cells.

The present invention further provides a composition for the treatment and/or prevention of intestinal inflammatory bowel diseases containing a Gram positive bacteria composition prepared by the following steps:
a) harvesting a culture of live bacteria cells,
b) washing the bacteria cells in water or in an aqueous solution of a salt such as borate,
c) freezing the bacteria cells in water or in an aqueous solution of a salt such as borate,
d) killing the frozen bacteria cells by drying them in a freeze-dryer, for a time sufficient to remove at least 98.5% of the water, preferably at least 99% of the water, more preferably at least 99.5% of the water, and
e) collecting the extended freeze-dried bacteria cells.

### BRIEF DESCRIPTION OF THE FIGURES

FIG. 1 illustrates changes in body weight and anus inflammation after addition of 2.5% DSS in drinking water during 7 days (C57BI/6 male mice).

FIG. 2 illustrates a difference in length of colon between mice receiving DSS in their drinking water and control (no DSS feeding) mice. The experimental model: C57BI/6 male mice received 2.5% of dextran sodium sulphate (DSS) in their drinking water during 7 days. Colon was collected on day 10 after the beginning of DSS feeding.

FIG. 3 illustrates the difference in length of the colon between EFD treated and untreated mice. Mice received 2.5% DSS in their drinking water during 7 days. A group of mice were EFD treated: 100 μg given subcutaneously 21 days before DSS feeding; or 1 mg per os at days 21, 20, 17 and 16 before DSS feeding. Colon was collected on day 10 after the beginning of DSS feeding.

FIG. 4 illustrates the difference in length of colon between the control group (no EFD treatment and no DSS feeding), the EFD treated mice (DSS feeding) and untreated mice (DSS feeding). The colons of EFD treated mice were similar to those of control mice. They were measured between caecum and anus.

FIG. 5 illustrates prevention of inflammatory bowel disease by EFD treatment. The colons of EFD treated mice were similar to those of control mice (no EFD and no DSS) (*** means p<0.001 with ANOVA statistical test). They were measured at day 8 or 10 i.e. 1 or 3 days after the end of DSS feeding.

FIG. 6 illustrates prevention of inflammatory bowel disease by EFD treatment. EFD treated mice did not develop a serious diarrhea or no diarrhea whereas the controls (DSS treated) did.

FIG. 7 illustrates a histological cut of the colon of a control (no DSS feeding, no EFD treatment) mice.

FIG. 8 is an enlarged view of a portion of the picture in FIG. 7.

FIG. 9 illustrates a histological cut of the colon of a mouse feeded with DSS.

FIG. 10 illustrates an enlarged view of a portion of the picture in FIG. 9.

FIG. 11 illustrates the histological cut of the colon of a mouse treated with EFD before DSS feeding.

FIG. 12 illustrates en enlarged view of a portion of the picture in FIG. 11.

FIG. 13a illustrates a histological cut of the colon of a control mouse.

FIG. 13b illustrates a histological cut of a colon of a mouse feeded with DSS.

FIG. 13c illustrates a histological cut of a colon of a mouse treated with EFD before DSS.

FIGS. 14a, b and c are enlarged views of FIGS. 13a, b and c respectively.

FIG. 15 illustrates the preliminary experiment with TNBS. C57BI/6 male mice received or not TNBS (1 mg in 100 μl of ethyl alcohol at 50%) twice, locally in colon at day −5 and 0. Their stools were observed during 10 days. At day 10, the mice were weighted. A group received EFD 100 μg subcutaneously 5 days before the first TNBS delivery (i.e. at day −10). A second group received PBS. The control group did not receive TNBS neither EFD. Weights in grams are reported on the figure (y-axis).

FIG. 16 illustrates the effects of EFD on DSS induced IBD in mice of protocol 2 during the 10 days of observation: a) on weight and b) on anus inflammation and stools. According to protocol 2, C57BI/6 male mice received or not 2.5% of dextran sodium sulfate (DSS) in their drinking water during 5 days. A group received EFD 100 μg subcutaneously 21 days before DSS. A group received PBS. The last group, control, did not drink DSS. This treatment protocol is the same for FIG. 16 and following FIGS. 17 to 24. On x-axis day 0 corresponds to the first day of DSS feeding. The pre-treatment with EFD decreases IBD symptoms.

FIG. 17 illustrates the effects of EFD pre-treatment on DSS feeded mice of protocol 2, as assessed by colon and caecum length. Colon and caecum lengths were measured at day 10 after the beginning of DSS fedding. Important inflammatory reaction due to DSS feeding conducted to a thickening of intestinal walls and a decrease in colon length of PBS treated mice, the colon weight being unchanged. The colons and caecums of EFD treated mice were similar to those of control mice (*** means p<0.001 with ANOVA statistical test).

FIG. 18 illustrates the effects observed at day 10 post the beginning of DSS feeding on mesenteric lymph node cell number. Mesenteric lymph nodes were collected, dissociated, and their cell contents were determined. An increased number of cells (×2.5) was observed. This increase was marginal when mice had been previously EFD treated (*** means p<0.001 with ANOVA statistical test).

FIG. 19 illustrates EFD effects on cytokines and lymphokines present in colonic tissues (a). Samples of colonic tissues were collected, weighed, dissociated in presence of protease inhibitors at day 10 post the beginning of DSS feeding. Their contents in different cytokines and lymphokines were determined. For IL-12p40 and RANTES no statistically significant differences were observed between PBS and EFD treated mice.

FIG. 20 illustrates EFD effects on cytokines and lymphokines present in colonic tissues (b). For IL-1β, TNF-α and MIP-1α, all implicated in inflammatory processes, statistically significant differences were observed between PBS and EFD treated mice, thus EFD treatment prevented the inflammation induced by DSS feeding.

FIG. 21 illustrates EFD effects on cytokines and lymphokines present in colonic tissues (c). IL-17, produced by activated T-cells, stimulates different cell lineages to produce inflammatory and hematopoietic cytokines. Less activated T-cells were present (according to IFN-γ measured quantity) and less IL-17 production was observed after EFD treatment.

FIG. 22 illustrates EFD effects on cytokines and lymphokines present in colonic tissues (d). KC, murin equivalent of IL-8, IL-6 and IL-1α are “inflammatory” cytokines or implicated in the NFκB signalling pathway. Their production was decreased after EFD treatment.

FIG. 23 illustrates EFD effects on cytokines and lymphokines present in colonic tissues (e). Hematopoeitic cytokines, IL-3, GM-CSF and G-CSF, were less produced after EFD treatment as suggested by preceding results showing a decreased IL-17 production.

FIG. 24 illustrates the fact that the increase in GATA-3 protein level observed in the spleen of mice drinking DSS supplemented water is prevented by EFD treatment. T-bet protein was highly produced in spleen 30 days after EFD treatment. Two samples, a and b, from DSS feeded mice were processed.

FIG. 25 illustrates the difference in body weight between EFD treated and untreated mice receiving dextran sodium sulphate (DSS) three times in their drinking water some days after EFD treatments. This model explored EFD preventive treatments in chronic IBD. Mice (C57BI/6 male mice) received 1.5% DSS in their drinking water during 7 days, ordinary tap water during 8 days, 1.5% DSS in their drinking water during 5 days, tap water during 10 days, 1.5% DSS during 5 days and tap water thereafter. A group of mice were EFD treated: 100 μg given subcutaneously 21 days before the first day of DSS feeding; or 1 mg per os at days 23, 22 and 21 before the first DSS feeding. They did not receive more EFD treatment. The weight of individual mouse was checked each day, 5 days a week. The experiment ended at day 52.

FIG. 26 illustrates the colitis score performed on mice included in the preventive assay (FIG. 25). Scores were checked according to:

0: no modification of feces

1: anus Inflammation

2: anus inflammation plus soft stool

3: anus inflammation plus diarrhoea

4: anus inflammation plus bloody diarrhoea

FIG. 27 up illustrates the difference in length of colon, measured between caecum and anus, at day 52 on mice included in the preventive assay (FIG. 25). The colons of EFD treated mice were similar to those of naive mice never fed with DSS (** means p<0.01 and *** means p<0.001 with ANOVA statistical test).

FIG. 27 down illustrates the number of cells found in the mesenteric lymph nodes collected at day 52 on mice included in the preventive assay (FIG. 25). The number of cells were decreased in the lymph nodes of EFD treated mice compared to those of PBS treated mice (*** means p<0.001 with ANOVA statistical test).

FIG. 28 illustrated the spleen weight and number of spleen cells at day 52 on mice included in the preventive assay (FIG. 25). The differences were not statistically different.

FIG. 29 illustrated the spontaneous release of some inflammatory cytokines by spleen cells collected at day 52 from mice included in the preventive assay (FIG. 25). 2×10⁵ cells were incubated in 0.2 ml of tissue culture medium during 96 h at 37° C., the supernatant were collected and the concentrations in IFN-γ, IL-6 and IL-17 were determined using the Multiplex BioRad assay. The differences observed after PBS and EFD treatments were extremely significant, whatever the cytokine, but much marked for IL-17.

FIG. 30 up illustrated the concentration of the transcription factor NFκB. NFκB was measured using a kit sell by Active Motif and used according to manufacturer protocol. This transcription factor was measured as optical density on 5 μg of nuclear extracts of spleen cells collected at day 52 from mice included in the preventive assay (FIG. 25). The differences observed after PBS and EFD treatments were highly significant (** means p<0.01 with ANOVA statistical test) or extremely significant (*** means p<0.001 with ANOVA statistical test).

FIG. 30 down illustrated the concentration of the transcription factor PPARγ. PPARγ was measured using a kit sell by Active Motif and used according to manufacturer protocol. This transcription factor was measured as optical density on 5 μg of nuclear extract of spleen cells collected at day 52 from mice included in the preventive assay (FIG. 25). The differences observed after PBS and EFD treatments were highly (** means p<0.01 with ANOVA statistical test) or extremely significant (*** means p<0.001 with ANOVA statistical test).

FIG. 31 illustrated the difference in body weight between EFD treated and untreated mice which have received dextran sodium sulphate (DSS) during 5 days in their drinking water. This model explored EFD curative treatment in acute IBD. Mice (C57BI/6 male mice) received 2.5% DSS in their drinking water during 5 days, and tap water thereafter. A group of mice were EFD treated: 100 μg given subcutaneously 24 hours after the last day of DSS feeding i.e. at day 6; or 1 mg per os at days 6, 7, and 8 after the 1^{st} day of DSS feeding. They did not receive more EFD treatment. The weight of individual mouse was checked each day, 5 days a week. The experiment ended at day 34. After EFD treatment, a slight, significant, curative effect was observed on day 13 and 15 (* p<0.05) with a faster recovery of weight loss.

FIG. 32 illustrates the colitis score performed on mice included in the curative assay (FIG. 31). Scores were checked according to previous description (FIG. 26). The clinical symptoms decreased faster in EFD treated mice.

FIG. 33 up illustrates the difference in length of colon, measured between caecum and anus, at day 34 on mice included in the curative assay (FIG. 31). The colons of EFD treated mice were similar to those of naive mice never fed with DSS (*** means p<0.001 with statistical ANOVA test).

FIG. 33 down illustrates the number of cells found in the mesenteric lymph nodes collected at day 34 on mice included in the curative assay (FIG. 31). The number of cells were decreased in the lymph nodes of EFD treated mice compared to those of PBS treated mice, the difference were statistically significant.

FIG. 34 illustrated the spleen weight (up) and number of spleen cells (down) at day 34 on mice included in the curative assay (FIG. 31). The differences were not statistically significant.

FIG. 35 illustrated the spontaneous release of some inflammatory cytokines by spleen cells collected at day 34 from mice included in the curative assay (FIG. 31). 2×10⁵ cells were incubated in 0.2 ml of tissue culture medium during 96 h at 37° C., the supernatant were collected and the concentrations in IFN-γ, IL-6 and IL-17 were determined using the Multiplex BioRad assay. The differences observed after PBS and EFD treatments were extremely significant (p<0.001) for IL-17, not significant for IFN-γ and IL-6.

FIG. 36 up illustrated the concentration of NFκB measured as optical density on 5 μg of nuclear extracts of spleen cells collected at day 34 from mice included in the curative assay (FIG. 31). The differences observed after PBS and EFD treatments were extremely significant (*** means p<0.001 with statistical ANOVA test).

FIG. 36 down illustrated the concentration of PPARγ measured as optical density on 5 μg of nuclear extract of spleen cells collected at day 34 from mice included in the curative assay (FIG. 31). The differences observed after PBS and EFD treatments were extremely significant (*** means p<0.001 with statistical ANOVA test).

FIG. 37 illustrates the difference in body weight between EFD treated and untreated mice receiving dextran sodium sulphate (DSS) three times in their drinking water some days and EFD treatments after the first series of DSS. This model explored EFD curative and preventive treatments in chronic IBD. Mice (C57BI/6 male mice) received 1.5% DSS in their drinking water during 7 days, ordinary tap water during 8 days, 1.5% DSS in their drinking water during 5 days, tap water during 10 days, 1.5% DSS during 5 days and tap water thereafter. A group of mice were EFD treated: 100 μg given subcutaneously 9 days after the first DSS feeding; or 1 mg per os at days 9, 10 and 11 after the first DSS feeding. They did not receive more EFD treatment. The weight of individual mouse was checked each day, 5 days a week. The experiment ended at day 52. After EFD treatment, a slight, significant, curative effect was observed on days 13, 15 with a faster and stable recovery of weight loss.

FIG. 38 illustrates the colitis score performed on mice included in the curative and preventive assay (FIG. 37). Scores were checked according to previous description (FIG. 26). The clinical symptoms decreased in subcutaneously EFD treated mice.

FIG. 39 up illustrates the difference in length of colon, measured between caecum and anus, at day 52 on mice included in the curative and preventive assay (FIG. 37). The colons of EFD treated mice were grossly similar to those of PBS mice.

FIG. 39 down illustrates the number of cells found in the mesenteric lymph nodes collected at day 52 on mice included in the curative and preventive assay (FIG. 37). The number of cells were decreased only in the lymph nodes of EFD treated subcutaneously (*** means p<0.001 with statistical ANOVA test).

FIG. 40 illustrated the spleen weight (up) and number of spleen cells (down) at day 52 on mice included in the curative and preventive assay (FIG. 37). The spleen weight and spleen cell number were decreased only in mice which have been subcutaneously EFD treated (* means p<0.05 and *** means p<0.001 with statistical ANOVA test).

FIG. 41 illustrated the spontaneous release of some inflammatory cytokines by spleen cells collected at day 52 from mice included in the preventive and curative assay (FIG. 37). 2×10⁵ cells were incubated in 0.2 ml of tissue culture medium during 96 h at 37° C., the supernatant were collected and the concentrations in IFN-γ, IL-6 and IL-17 were determined using the Multiplex BioRad assay. The differences observed after PBS and EFD treatments were extremely significant (p<0.001) whatever the cytokine, but much marked for IL-17.

FIG. 42 up illustrated the concentration of NFκB measured as optical density on 5 μg of nuclear extracts of spleen cells collected at day 52 from mice included in the curative and preventive assay (FIG. 37). The differences observed after PBS and EFD treatments were extremely significant (*** means p<0.001 with statistical ANOVA test).

FIG. 42 down illustrated the concentration of PPARγ measured as optical density on 5 μg of nuclear extract of spleen cells collected at day 52 from mice included in the curative and preventive assay (FIG. 37). The differences observed after PBS and EFD treatments were extremely significant (*** means p<0.001 with statistical ANOVA test).

FIG. 43 illustrates the difference in body weight between EFD treated and untreated mice receiving dextran sodium sulphate (DSS) three times in their drinking water and EFD treatments after the last series of DSS. This model explored EFD curative treatments in chronic IBD. Mice (C57BI/6 male mice) received 1.5% DSS in their drinking water during 7 days, ordinary tap water during 8 days, 1.5% DSS in their drinking water during 5 days, tap water during 10 days, 1.5% DSS during 5 days and tap water thereafter. A group of mice were EFD treated: 100 μg given subcutaneously 7 days after the last DSS feeding; or 1 mg per os at days 42, 43 and 45 after the first DSS feeding. The weight of individual mouse was checked each day, 5 days a week. The experiment ended at day 54. After subcutaneous EFD treatment, a slight, curative effect was observed on day 54 with a recovery of weight loss.

FIG. 44 illustrates the colitis score performed on mice included in the curative assay of chronic IBD (FIG. 43). Scores were checked according to previous description (FIG. 26). The clinical symptoms decreased in subcutaneously EFD treated mice.

FIG. 45 up illustrates the difference in length of colon, measured between caecum and anus, at day 54 on mice included in the curative assay of chronic IBD (FIG. 43). The colons of EFD treated mice were grossly similar to those of PBS mice (* means p<0.05 with statistical ANOVA test).

FIG. 45 down illustrates the number of cells found in the mesenteric lymph nodes collected at day 54 on mice included in the curative assay (FIG. 43). The number of cells were decreased only in the lymph nodes of EFD treated subcutaneously (*** means p<0.001 with statistical ANOVA test).

FIG. 46 illustrates the spleen weight (up) and number of spleen cells (down) at day 54 on mice included in the curative assay of chronic IBD (FIG. 43). The spleen weight and spleen cell number were decreased only in mice which have been subcutaneously EFD treated (* means p<0.05 and ** means p<0.01 with statistical ANOVA test).

FIG. 47 illustrates the spontaneous release of some inflammatory cytokines by spleen cells collected at day 54 from mice included in the curative assay of chronic IBD (FIG. 43). 2×10⁵ cells were incubated in 0.2 ml of tissue culture medium during 96 h at 37° C., the supernatant were collected and the concentrations in IFN-γ, IL-6 and IL-17 were determined using the Multiplex BioRad assay. The differences observed after PBS and EFD treatments were extremely significant (p<0.001) whatever the cytokine, but much marked for IL-17.

FIG. 48 up illustrates the concentration of NFκB measured as optical density on 5 μg of nuclear extracts of spleen cells collected at day 54 from mice included in the curative assay of chronic IBD (FIG. 43). The differences observed after PBS and EFD treatments were significant or extremely significant (* means p<0.05 and *** means p<0.001 with statistical ANOVA test).

FIG. 48 down illustrates the concentration of PPARγ measured as optical density on 5 μg of nuclear extract of spleen cells collected at day 54 from mice included in the curative assay of chronic (FIG. 43). The differences observed after PBS and EFD treatments were very or extremely significant (** means p<0.01 and *** means p<0.001 with statistical ANOVA test).

### DETAILED DESCRIPTION OF THE INVENTION

### Definitions

The expression “killed or non infectious Gram positive bacteria preparation” as used in the context of the present invention refers to a preparation of killed or non infectious Gram positive bacteria as described in WO03049752. This Gram positive bacteria preparation contains killed or non infectious Gram positive bacteria, obtainable by a process which does not denature the structure of the molecules contained therein and particularly the proteins contained therein. Advantageously, this Gram positive bacteria preparation contains extended-freeze-dried killed bacteria and less than 1.5% of residual water, preferably less than 1%, more preferably less than 0.5%. These extended-freeze-dried killed bacteria are prepared by harvesting a culture of live bacteria cells, washing the bacteria cells in water or in an aqueous solution of a salt such as borate, freezing the bacteria cells in water or in an aqueous solution of salt such as borate, killing the frozen bacteria cells by drying them in a freeze-dryer, for a time sufficient to remove at least 98.5% of the water, preferably at least 99% of the water, more preferably at least 99.5% of the water, and collecting the extended-freeze-dried killed bacteria cells.

A fraction of this extended-freeze-dried killed bacteria preparation is covered by the expression “Gram positive bacteria preparation” of the invention. This Fraction is selected in the group consisting of: a fraction consisting of an organic solvent extract of said killed bacterial preparation, a fraction consisting of a glycosidase-treated extract of said killed bacterial preparation, a fraction consisting of a DNAse and/or RNase-digested extract of said killed bacterial preparation and a fraction consisting of said killed bacterial preparation successively treated by an organic solvent, a glycosidase, a DNase and/or RNase, and finally a protease.

The expression “intestinal inflammatory syndromes” as used in the context of the present invention relates to any inflammatory bowel diseases including the two entities Crohn's disease and ulcerative colitis and an intermediate variant of these diseases, indeterminate colitis which shows overlapping features of the two diseases previously enumerated.

### Use of the Gram Positive Bacteria Preparation

The invention also relates to the use of a Gram positive bacteria preparation for the prevention and treatment of intestinal inflammatory syndromes, the preparation being characterized in that the Gram positive bacteria are killed or non infectious, and containing more than 50%, and preferentially more than 90%, of the bacterial protein components which are in a native structure. The Gram positive bacteria preparation of the invention may also be useful for the preparation of a medicament for the prevention and/or treatment of intestinal inflammatory syndromes.

According to a preferred embodiment of the present invention, the Gram positive bacteria preparation is a Gram positive facultative intracellular bacteria. Gram positive facultative intracellular bacteria means Gram positive bacteria with a capacity of growing in synthetic medium in vitro as well as of infecting eucaryotic cells from a mammalian or non-mammalian host, in vivo and multiplying in those cells, for example, macrophages.

According to another preferred embodiment, the bacterial preparation contains Gram positive facultative intracellular bacteria chosen from the group consisting of Listeria sp., Corynobacterium sp. and Actinomycetes comprising Mycobacteria sp., Nocardia sp. and Rhodococcus sp.

More preferably, the bacterial preparation contains Mycobacterium bovis and even more preferably, Mycobacterium bovis BCG.

The instant invention also relates to the use of the killed or non infectious bacteria preparation or fractions thereof for the preparation of a medicament for the prevention and/or treatment of a disease comprising an immune dysregulation such as a Th1/Th2 imbalance. According to a preferred embodiment of the invention, the disease is Crohn's disease or ulcerative colitis.

The killed bacteria preparation or fractions thereof may be associated with a pharmaceutically acceptable carrier, and/or an immunostimulant, and/or an adjuvant and/or any conventional additives as defined herein below. The Gram positive bacteria preparation and/or the medicament of the invention may be administered by the oral, sublingual, parenteral or intranasal route.

### Pharmaceutical Compositions

As mentioned herein above, the present invention relates to the use of a killed or non infectious Gram positive bacteria preparation for the preparation of a pharmaceutical composition for the prevention and/or treatment of intestinal inflammatory disorders.

According to an embodiment of the invention, such a composition is obtained by:
a) harvesting a culture of live bacteria cells,
b) washing the bacteria cells in water or in an aqueous solution of a salt such as borate,
c) freezing the bacteria cells in water or in an aqueous solution of a salt such as borate,
d) killing the frozen bacteria cells by drying them in a freeze-dryer, for a time sufficient to remove at least 98.5% of the water, preferably at least 99% of the water, more preferably at least 99.5% of the water, and
e) collecting the extended freeze-dried bacteria cells.

The composition of the present invention is preferably in a form suitable for oral administration. For example, the composition may be in the form of tablets, ordinary capsules, gelatine capsules or syrup for oral administration. These gelatine capsules, ordinary capsules and tablet forms can contain excipients conventionally used in pharmaceutical formulations such as adjuvants or binders like starches, gums and gelatine, adjuvants like calcium phosphate, disintegrating agents like corn starch or algenic acids, a lubricant like magnesium stearate, sweeteners or flavourings. Solutions or suspensions can be prepared in aqueous or non-aqueous media by the addition of pharmacologically compatible solvents. These include glycols, polyglycols, propylene glycols, polyglycol ether, DMSO and ethanol.

According to another preferred embodiment, the composition of the present invention is preferably in a form suitable for parenteral administration, such as subcutaneous injection. For parenteral administration, such as subcutaneous injection, the carrier preferably comprises water, saline buffer, lactose, glutamate, a fat or a wax. For oral administration, any of the above carriers or a solid carrier such as mannitol, lactose, starch, magnesium stearate, sodium saccharine, talcum, cellulose, glucose, sucrose and magnesium carbonate may be employed. Biodegradable microspheres (e.g. polylactic galactide) may also be employed as carriers for the pharmaceutical compositions of this invention. Suitable biodegradable microspheres are disclosed for example in U.S. Pat. Nos. 4,897,268 and 5,075,109.

The compositions of the invention may additionally contain an additive and/or an immunostimulant and/or an adjuvant such as a liposome containing the bacteria cells or fractions thereof according to the present invention. The additives used for preparing the pharmaceutical composition of the present invention may be chosen among anti-aggregating agents, antioxidants, dyes, flavour enhancers, or smoothing, assembling or isolating agents, and in general among any excipient conventionally used in the pharmaceutical industry.

Any of the variety of adjuvants may be employed in the compositions of the present invention to enhance the immune response. Most adjuvants contain a substance designed to protect the antigen from rapid catabolism or to create controlled inflammatory reactions, such as aluminium hydroxide or mineral oil, and a non-specific stimulator of immune response such as lipid A, Bordetella pertussis toxin. Suitable adjuvants are commercially available as well, for example, Freund's incomplete adjuvant and Freund's complete adjuvant which cannot be used for injection in human. Other suitable adjuvants which can be used in human include aluminium hydroxide, biodegradable microspheres, monophospheryl A and Quil A.

### Methods of Prevention or Treatment

According to an embodiment of the present invention, the killed or non infectious Gram positive bacteria preparation is used for the prevention and treatment of intestinal inflammatory disorders selected from the group consisting of Crohn's disease and ulcerative colitis. The method of the invention comprises the step of administering to a patient an effective amount of the killed or non infectious Gram positive bacteria preparation, to stimulate the production of leukocytic regulatory cells, such as CD4+, CD25+, T cells, B cells and/or dendritic cells.

According to another embodiment of the present invention, there is provided a method for preventing or treating a disease caused by a Th1/Th2 imbalance. The method comprises the steps of a) providing a killed or non infectious Gram positive bacteria preparation or a portion thereof retaining the capacity of inhibiting intestinal inflammatory syndromes or a pharmaceutical composition of the invention and b) administering an effective amount of the Gram positive bacteria preparation to a patient affected by the disease.

As mentioned above, the intestinal inflammatory syndrome may be Crohn's disease or ulcerative colitis.

The amount of Gram positive bacteria preparation present in the compositions of the present invention is preferably a therapeutically effective amount. A therapeutically effective amount of Gram positive bacteria preparation is that amount necessary so that the Gram positive bacteria preparation performs its role of inhibiting intestinal inflammatory syndrome without causing, overly negative effects in the host to which the composition is administered. The exact amount of Gram positive bacteria preparation to be used and the composition to be administered will vary according to factors such as the type of intestinal inflammatory syndrome being treated, the mode of administration, as well as the other ingredients in the composition. Preferably, the composition is composed of from about 10 μg to about 10 mg and more preferably from about 100 μg to about 1 mg, of killed or non infectious Gram positive bacteria preparation. By “about”, it is meant that the value of said quantity (μg or mg) of killed or non infectious Gram positive bacteria preparation can vary within a certain range depending on the margin of error of the method used to evaluate such quantity.

For instance, during an oral administration of the composition of the invention, host to be treated could be subjected to a 1 dose schedule of from about 10 μg to about 10 mg of killed or non infectious Gram positive bacteria preparation per day during 3 consecutive days. The treatment may be repeated once one week later.

For parenteral administration, such as subcutaneous injection, the host to be treated could be subjected to a 1 dose schedule of from about 10 μg to about 10 mg and more preferably from about 100 μg to about 1 mg, of killed or non infectious Gram positive bacteria preparation per month or every 6 months.

### Method of Preparation of the Gram Positive Bacterial Preparation

For instance, the Gram positive bacteria of the present invention may be killed by “soft methods” which do not denature the molecules from the bacteria cells and thus are able to stimulate leukocyte regulatory cells (CD4+, CD25+, T cells and/or B cells and/or dendritic cells) in vivo when they are administered to subjects suffering from immune dysregulation. Thus, the Gram positive bacteria preparation according to the present invention may consist of heat-killed preparations of Mycobacteria. According to a preferred embodiment of the present invention, the Gram positive bacteria are killed by lyophilization. These processes which are denominated soft processes include with no limitation the use of physical means which disrupt the bacteria cell membranes while preserving the structure of its macromolecular components. These processes include with no limitation: extended freeze-drying, grinding in the presence of silica or zirconium beads, use of a so-called “French press”, sonification and gamma-rays irradiation. Other processes which may be used for obtaining the killed bacteria preparation as defined above are known to those of ordinary skill in the art.

A process which does not denature the structure of the molecules from the bacteria cells means a process which results in no extensive denaturation of a configuration of the molecules. Preferably, such process preserves the three dimensional structure of the micromolecules from the bacteria cells such as proteins, polysaccharides and lipids.

### EXAMPLES

### General Information

To test the capacity of EFD to decrease inflammatory reactions in IBD, two mouse models of IBD were explored:

IBD secondary to TNBS local sensitisation

IBD secondary to DSS feeding.

### IBD Secondary to TNBS Local Sensitisation

TNBS (2,4,6-trinitrobenzene sulfonic acid) is a chemical containing a phenol derivative which creates a “pure” T-lymphocyte dependent sensitization. The sensitization is performed by local delivery into the colon of a TNBS solution through a small tube inserted in the anal canal. Some days later the local delivery of the same chemical creates local inflammatory lesions with ulcerative aspects due to the local T-lymphocyte response.

The advantage of this model is to explore T-lymphocyte dependent inflammatory reactions.

The disadvantages of this model are the following:

each mouse has to be anesthetised twice, to be carefully handle to introduce a tiny tube into the anus

TNBS is a potent sensitizer for human beings. The risks to sensitize laboratory workers are high, knowing that equivalent phenol derivatives are frequent in our environment. It has to be handle with high precocious.

### IBD Secondary to DSS Feeding

DSS (Dextran sodium sulphate) is a chemical which creates local intestinal lesions in mice when ingested. Its delivery via drinking water is easy. It is a less potent sensitizer than TNBS, with fewer risks for the laboratory workers. The mechanisms of lesions are partly understood, the absence of DSS-induced lesions in germ-free mice excludes a direct, single, immunological mechanism. The necessity for the presence of intestinal bacteria being also observed in the Crohn's disease in human, DSS model was proposed by some researchers to be closer than TNBS model to human diseases.

### Example I

### Preventive Model of DSS Induced IBD (Short Term Effect)

### Protocols

### Protocol 1

C57BI/6 mice, 6 to 7 weeks old, were distributed in 4 groups of 10 mice:
Mice from Group 1 did not receive anything; they composed the control group.
Mice from Group 2 received 100 μl of isotonic saline solution subcutaneously. Their drinking water was replaced 21 days later by a 2.5% DSS solution in water for 7 days.
Mice from Group 3 were fed 1 mg of EFD on days 1 and 2, and again were fed 1 mg EFD on days 8 and 9. Their drinking water was replaced by the DSS solution on day 21 for 7 days.
Mice from Group 4 received 100 μg EFD in 100 μl saline solution subcutaneously at the base of the tail on day 1. Their drinking water was replaced by the DSS solution on day 21 for 7 days.

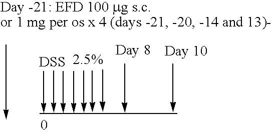

All mice were killed at day 8 (5 mice) or day 10 (5 mice) to analyse early pathological modifications: colon length, histological findings.

### Protocol 2

This protocol is similar to protocol 1. C57BI/6 male mice (per group of 10 mice) received or not 2.5% of dextran sodium sulfate (DSS) in their drinking water during 5 days. This protocol was slightly lighter than the protocol 1, DSS being given during 5 days instead of 7 days. A group received EFD 100 μg subcutaneously 21 days before DSS. A group received PBS instead of EFD. The last group, control, receive neither DSS nor EFD.

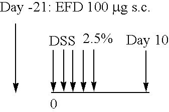

Daily weighing and observation of the animals were made during the 8 or 10 days after the beginning of DSS ingestion. A clinical score summarizing the intensity of the observed signs was established:

0, normal mouse

1, inflammation of the anus

2, inflammation of the anus and soft stools

3, inflammation of the anus and diarrhea

4, inflammation of the anus and bloody diarrhea

On the 8^{th} day after the beginning of DSS ingestion, 4 animals of each group were sacrificed, their colon was collected and measured (length between the anus and the caecum), placed in Tissue-Tek O.C.T. (Sakura Finetek) and frozen for histological examination. The surviving mice were sacrificed on day 10.

Samples on colonic tissues were collected on day 10 in order to determine their content in different cytokines/lymphokines using a Multiplex kit (BioRad).

### Results

### 1. Weight Curves and Clinical Appearance of Mice

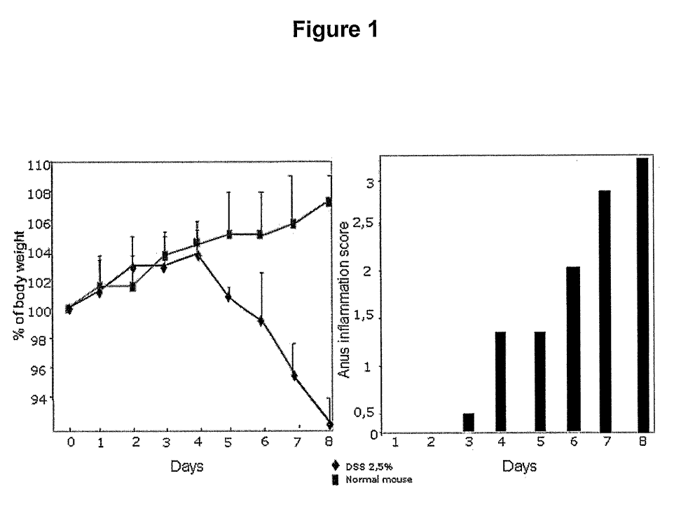

The mice not treated with EFD receiving the dextran sodium sulphate solution as drinking water, Group 2, lose weight rapidly (FIG. 1 left side). The vivaciousness of the animals is very reduced on the 6^{th} or 7^{th} day of this ingestion; they do not recuperate at all or do not recuperate well 24 or 72 hours after stopping DSS ingestion. 3 mice died on day 8.

The mice treated with EFD, per os or subcutaneously, from Groups 3 and 4 appear sick, but in a less important manner; they remain vivacious and appear susceptible to recuperation. There is no mortality among those groups.

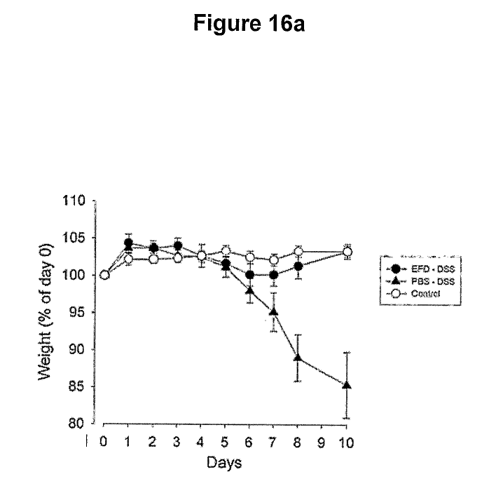

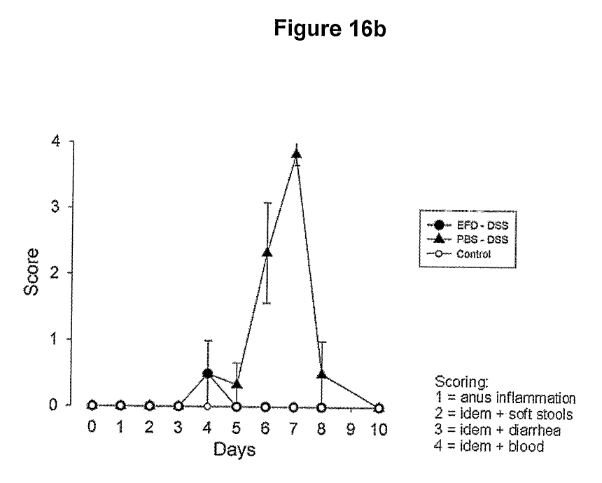

The group of mice treated with EFD according to protocol 2 showed a similar weight curve as the group of control mice (FIG. 16a), demonstrating that the pretreatment with EFD decreases IBD symptoms.

### 2. Clinical Scores

The mice that were not treated with EFD present an elevated clinical score (inflammation and diarrhea) after 4 or 5 days of ingestion of DSS. This score becomes increasingly elevated after 7 to 8 days (FIG. 1 right side).

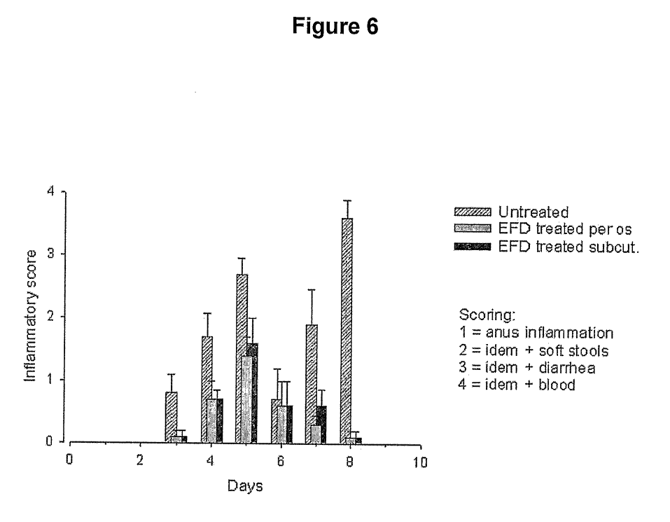

The mice treated with EFD present identical clinical scores when compared to each other, scores less elevated than those observed for the non-treated animals on days 4 and 5 after the beginning of DSS ingestion. This score regresses, and is then normalized. The mice having been treated with EFD do not present inflammation of the anus, nor diarrhea, on days 7 and 8 (FIG. 6). Mice having been treated with EFD according to protocol 2 showed clinical scores similar to the ones of control mice (FIG. 16b).

### 3. Inflammation of Colon

a) Macroscopic Examination

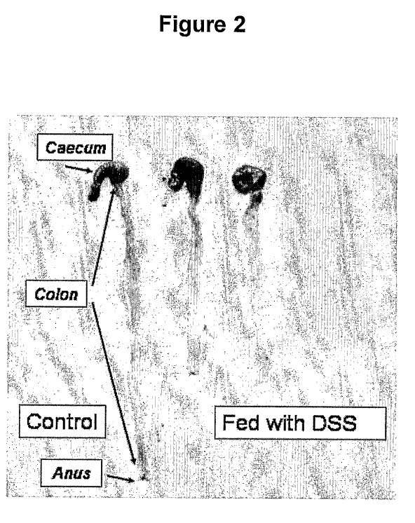

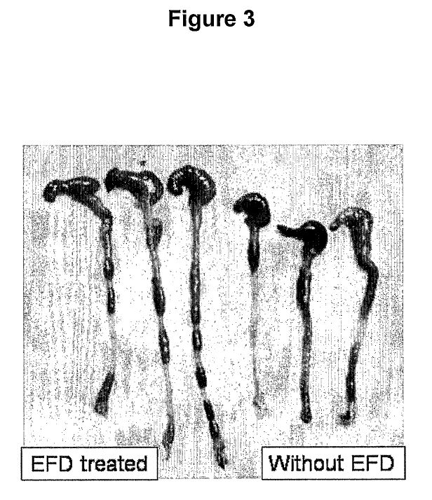

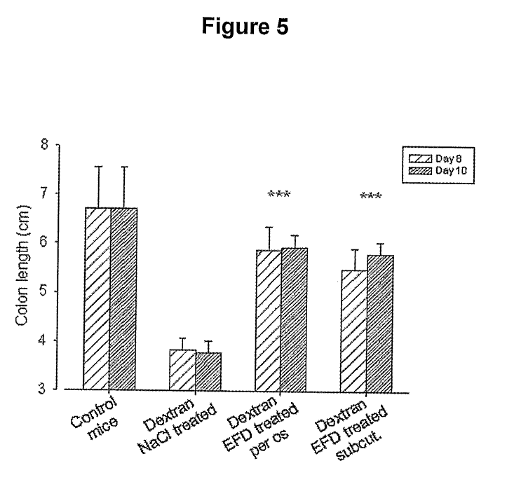

The animals were sacrificed 8 or 10 days after the beginning of DSS ingestion, either 1 or 3 days after stopping such ingestion. No difference was observed depending on the date of autopsy. The colon of the control animals contained many feces which became increasingly harder with their migration towards the anus. The average length of the colon is 6.7 cm (FIG. 5). The length of the colon of the animals having received DSS is reduced to approximately 3.8 cm (FIGS. 2 and 5). The stools are soft and the colon very often hemorrhagic in DSS treated mice.

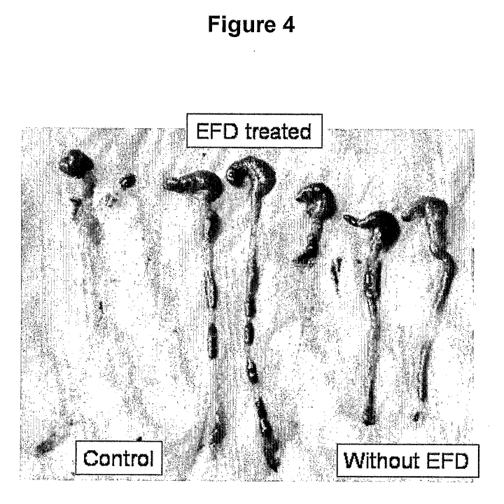

The colon of animals having been treated with EFD contained feces that were more or less hard, of identical appearance to those of the control animals (FIG. 4). The average length of the colon is of 5.8 cm (FIG. 5). No significant difference was observed in the effect of EFD treatments according to the mode of administration (FIG. 5).

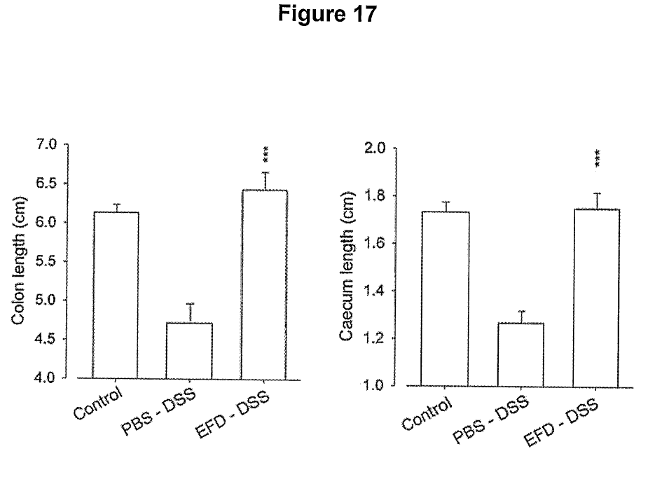

The mice having been treated with EFD in accordance with the protocol 2 showed colons and caecum of lengths similar to those of control mice never fed with DSS (FIG. 17).

### b) Microscopic Examination

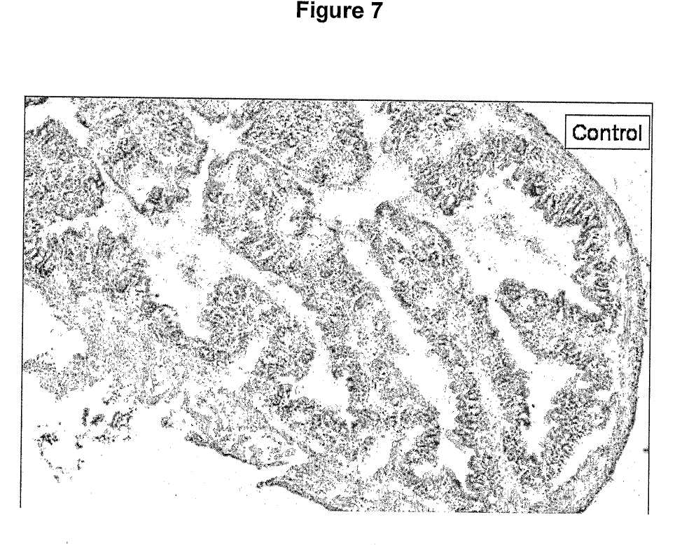

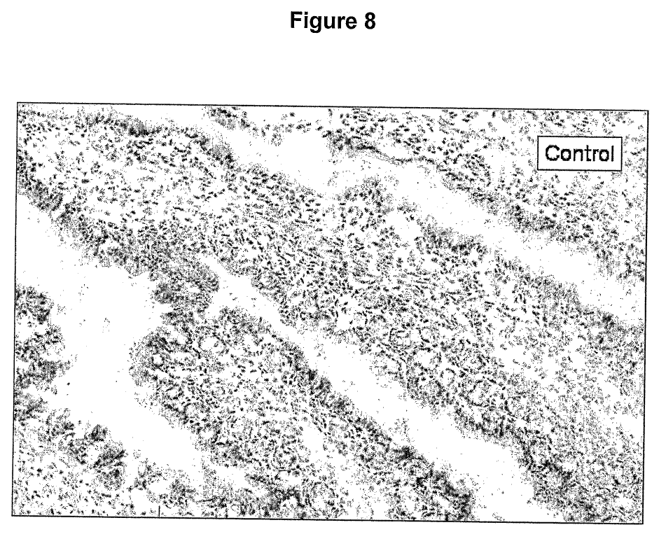

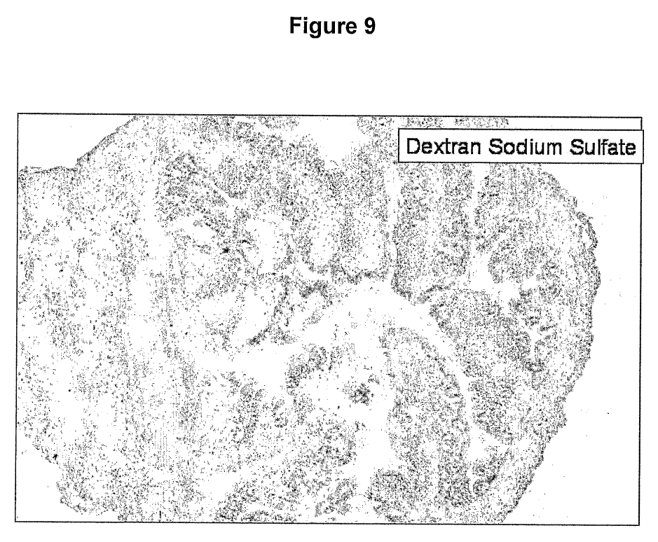

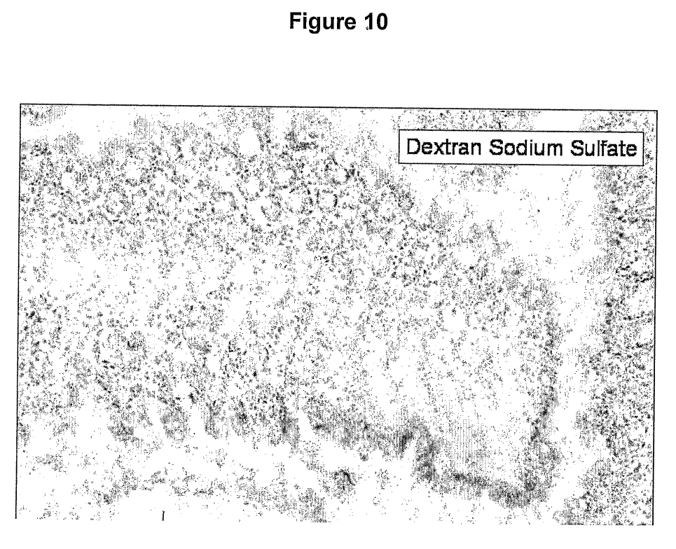

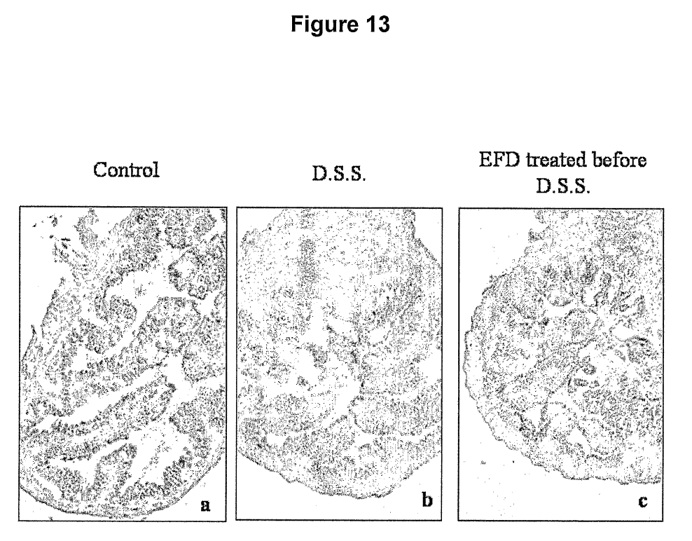

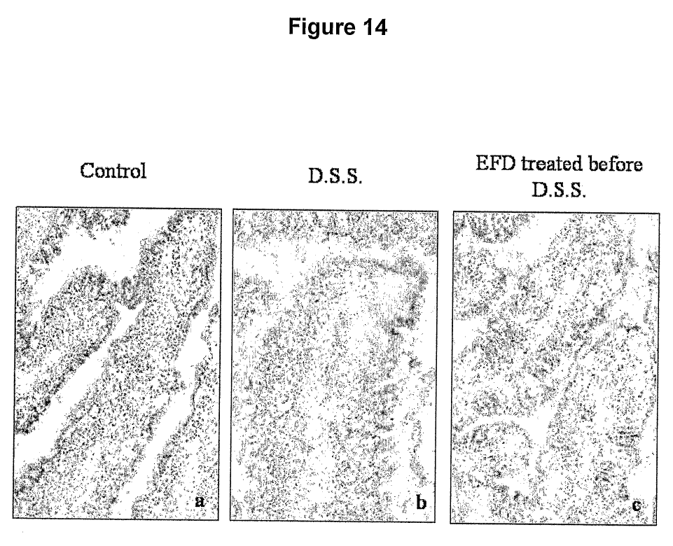

The microscopic examination of the lesions after the administration of DSS shows intense inflammatory responses at the level of the intestinal mucosa, an oedema of the sub-mucosa, important at the level of the insertion of the mesentery, associated to a thickening of the intestinal wall. The intestinal villi are dissociated and partially unstructured (FIGS. 9 and 10 versus FIGS. 7 and 8 for control, FIGS. 13 and 14).

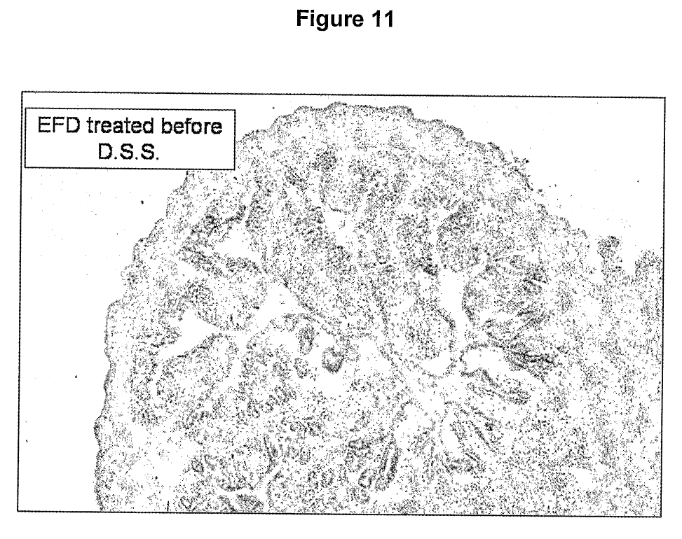

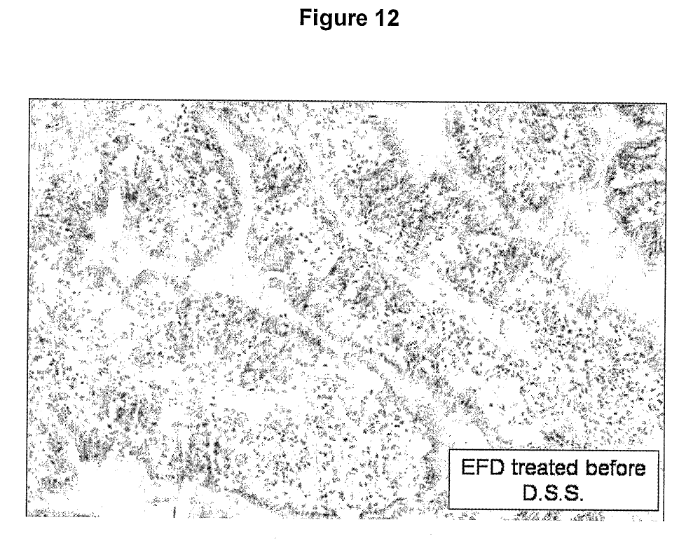

In the animals that were treated with EFD before the ingestion of DSS, the lesions are much less important (FIGS. 11 and 12 versus FIGS. 7 and 8 for control). The intestinal villi have a morphology close to that observed in the control animals (FIGS. 13 and 14).

### c) Inflammatory Regulators Present in Colonic Tissues and Spleen

Dosage of IL-12p40, IL-12p70, RANTES, IL-1 beta, TNF alpha, MIP-1 alpha, IL-17, IFN gamma, IL-10, KC, IL-6, IL-1 alpha, IL-3, GM-CSF, and G-CSF. All cytokines and lymphokines were measured using Bioplex method according to manufacturer recommendations (Bio-Rad).

Dosage of T-bet and GATA-3 proteins in spleen.

Total proteins extracted from the spleen cells were resolved on 7.5% SDS-PAGE, then protein bands transferred to nitrocellulose sheets were probed with polyclonal rabbit anti-mouse FOXP3 IgG kindly provided by E. Schmitt and C. Richter (Institute of Immunology, Mainz, Germany) with mouse monoclonal anti-T-bet, mouse monoclonal anti-GATA-3 (Santa Cruz Biotechnology, Santa Cruz, Calif.) or β-actin mouse monoclonal Ab (Ac-15 Abcam, Cambridge, UK). As secondary Ab, we used HRP-labeled polyclonal goat anti rabbit (Dako Cytomation, Denmark). The immune complex was revealed by enhanced chemiluminescence detection system (Amersham, France).

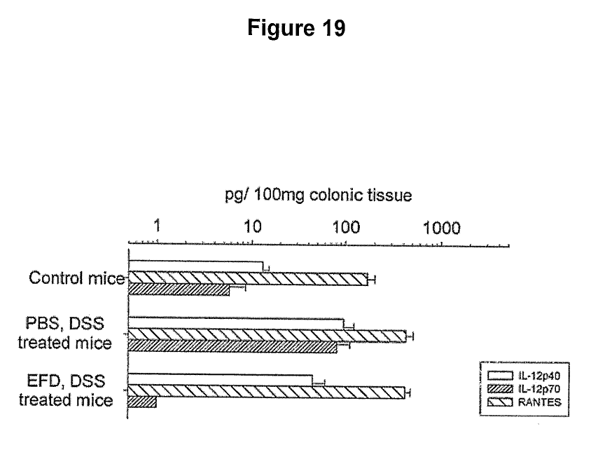

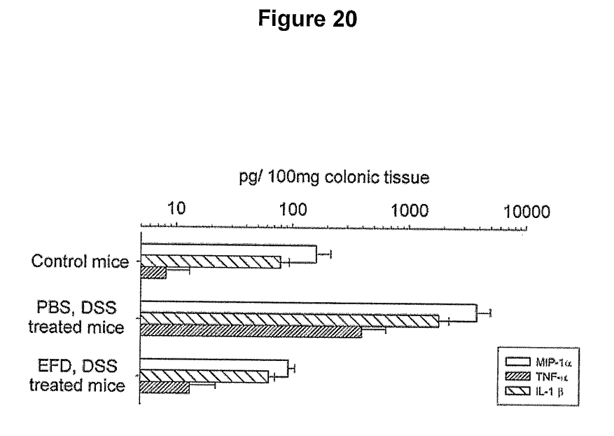

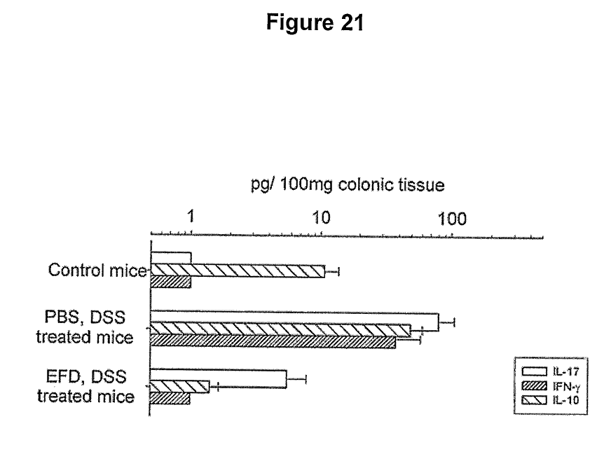

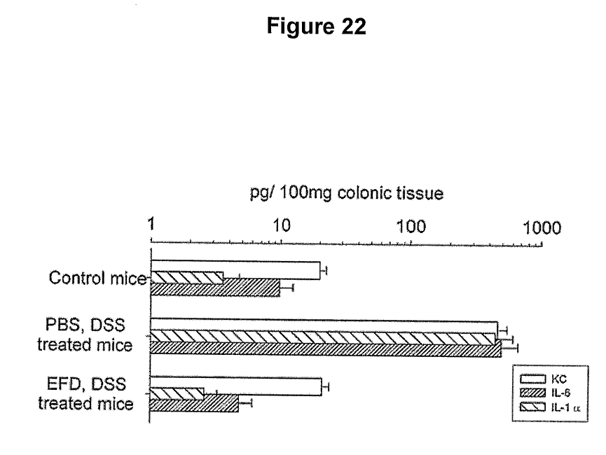

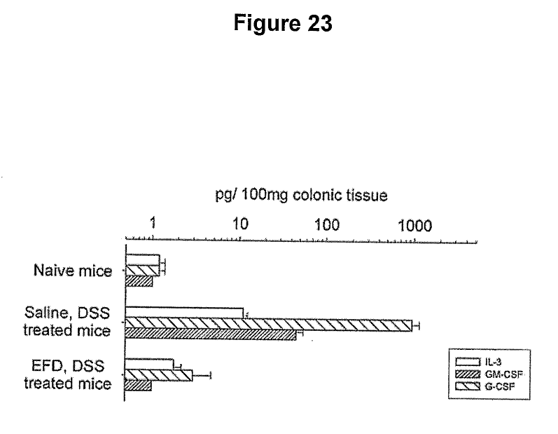

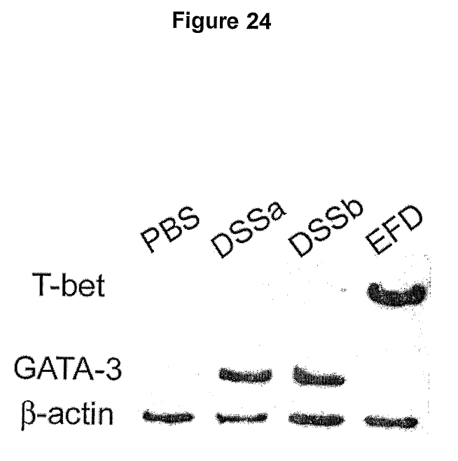

Inflammatory cytokines and lymphokines are present in high concentration in the colonic samples collected from mice having DSS in their drinking water. These molecules are the origin or the consequence of the important pathological changes observed in the mice. In the animals that were treated subcutaneously with EFD before the ingestion of DSS, the concentrations of these molecules were in the range observed for control animals receiving tap water without DSS (see FIGS. 19-24).

### d) Mesenteric Lymph Nodes Cell Number

An increase of the size of mesenteric lymph nodes is commonly associated with colonic disease and more particularly with inflammatory bowel disease.

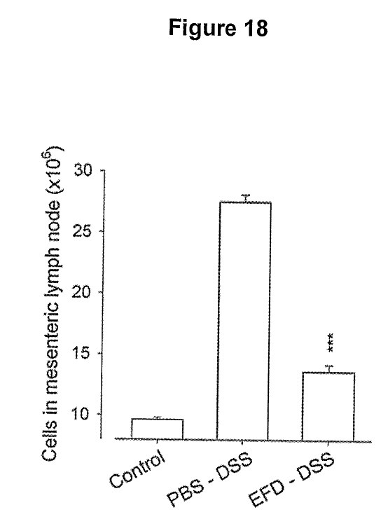

At day 10 post beginning of DSS feeding, mesenteric lymph nodes of mice from protocol 2 were collected, crushed on cell-strainers (Falcon). The dissociated cells were washed in AIM V medium (Gibco) supplemented with 5% FCS, centrifuged and the pelleted cells resuspended in 0.5 or 1 ml of medium. Cells were diluted 10 fold in trypan blue (0.1% in PBS) and counted under microscope in a Malassez cell. An increased number of cells (×2.5) is observed for mice PBS-treated and feeded with DSS in comparison with mice control. EFD preventive treatment permits to significantly reduce this number to the value of control mice (FIG. 18).

### Example II

### Preventive Model of DSS Induced Chronic IBD (Long Term Effect)

This model explored EFD preventive treatments in chronic IBD.

### Protocol

C57BI/6 mice, 6 to 7 weeks old, were distributed in 4 groups of 10 mice.

Mice (C57BI/6 male mice) received 1.5% DSS in their drinking water during 7 days, ordinary tap water during 8 days, 1.5% DSS in their drinking water during 5 days, tap water during 10 days, 1.5% DSS during 5 days and tap water thereafter. A group of mice were EFD treated: 100 μg given subcutaneously 21 days before the first day of DSS feeding; or 1 mg per os at days 23, 22 and 21 before the first DSS feeding. They did not receive more EFD treatment. The experiment ended at day 52.

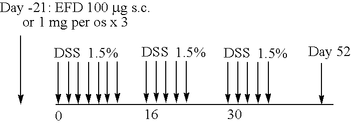

### Results

Weight Curves

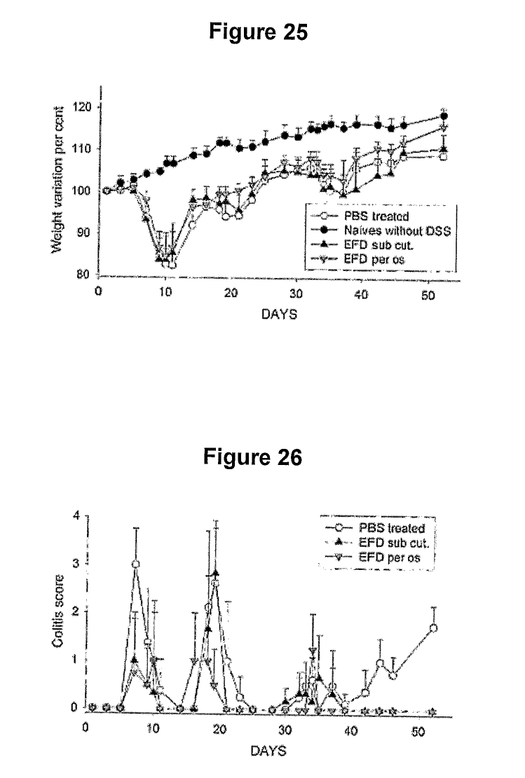

The mice treated with EFD, per os or subcutaneously, appear to lose weight and gain weight again in a similar manner than mice not treated with EFD and feeded with DSS but at the end of the experiment (day 50) after the three series of DSS ingestion, the preventive treatment permits to mice to have a weight very close to the one of mice control (FIG. 25).

Clinical Scores

According to FIG. 26, at the end of the experiment (day 50), mice not treated with EFD present a clinical score arising continuously after the end of DSS feeding. The EFD pretreatment permits mice to have a normalized clinical score at the end of experiment, when the mice not treated with EFD see their clinical scores continuously arise even after the three IBD simulations.

Inflammation of Colon

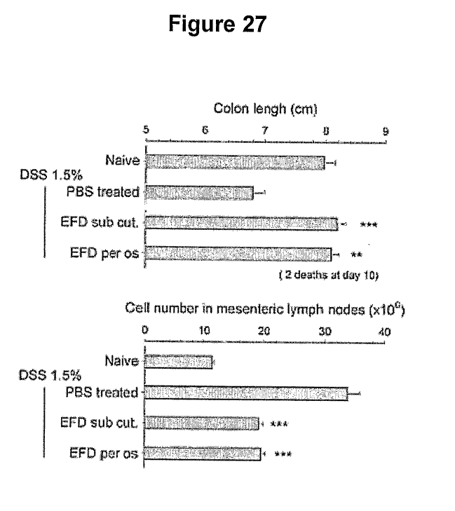

At the end of experiment (day 52), the colons of EFD treated mice were similar to those of naive mice never fed with DSS (FIG. 27 up).

Lymph Node and Spleen

At the end of experiment (day 52), the number of cells in the lymph nodes of EFD treated mice were decreased compared to those of PBS treated mice and can be expected to revert progressively to the cell number of control mice never fed with DSS (FIG. 27 down).

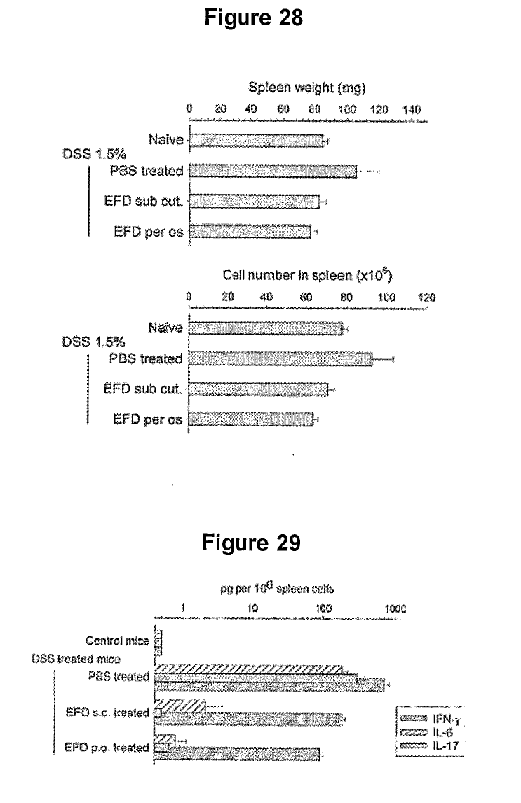

As illustrated in FIG. 28, the difference in spleen weight and number of spleen cells at day 52 on mice included in this preventive assay are not statistically different.

Inflammatory Regulators Release from Spleen

The spontaneous release of some inflammatory cytokines by spleen cells collected at the end of the experiment (day 52) was studied. The differences observed after PBS and EFD pretreatments were extremely significant whatever the cytokine (IFN gamma, IL-6 and IL-17) but much marked for IL-17. EFD pretreatment permits after chronic IBD to keep levels of cytokines release similar to the ones of naive mice never fed with DSS (FIG. 29).

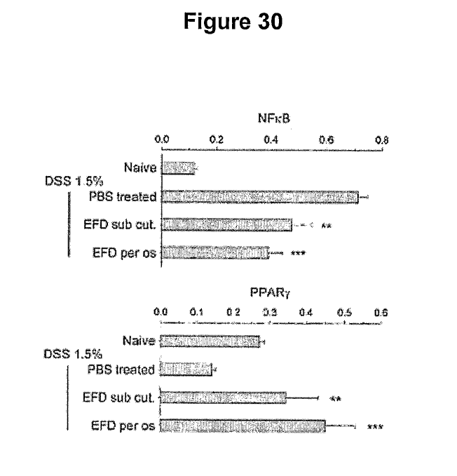

The nuclear factor-kappaB (NF-κB) is known to be activated in inflammatory bowel diseases and up-regulate inflammatory cytokines. As illustrated in FIG. 30 up, the high concentration of NF-κB provoked by recurrent DSS treatments of mice is significantly decreased by EFD pretreatment.

The peroxisome proliferator-activated receptor-γ (PPAR-γ), a member of the nuclear receptor superfamily of ligand-dependent transcription factors predominantly expressed in adipose tissue, adrenal gland and spleen is involved in the regulation of numerous inflammatory responses and in particularly of intestinal inflammation. It has been proposed as a key inhibitor of colitis though attenuation of nuclear factor kappa B (NF-κB) activity. Levels of PPAR-γ in spleen are decreased during chronic inflammation as illustrated by FIG. 30 down. The preventive treatment of mice with EFD permits to increase the level of PPAR-γ in chronic IBD model at and beyond the level of naive mice (without chronic IBD).

### Example III

### Curative Model of DSS Induced IBD (Long Term Effect)

This model explored EFD curative treatment in acute IBD.

### Protocol

C57BI/6 mice, 6 to 7 weeks old, were distributed in 4 groups of 10 mice. Mice (C57BI/6 male mice) received 2.5% DSS in their drinking water during 5 days, and tap water thereafter. A group of mice were EFD treated: 100 μg given subcutaneously 24 hours after the last day of DSS feeding (i.e. at day 6); or 1 mg per os at days 6, 7, and 8 after the 1^{st} day of DSS feeding. They did not receive more EFD treatment. The experiment ended at day 34.

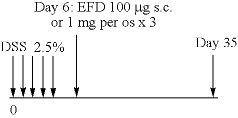

### Results

Weight Curves

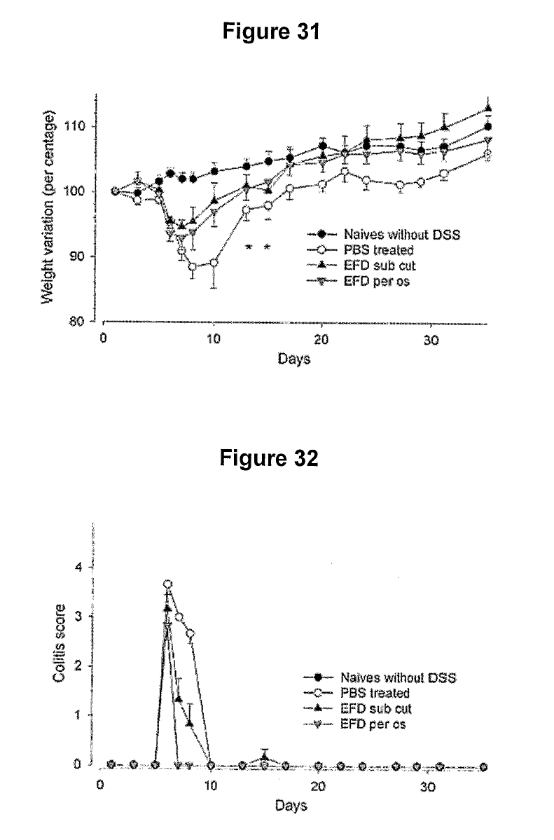

In this model of curative treatment in acute DSS-induced IBD, the weight of individual mouse was checked each day, 5 days a week from the first day of DSS ingestion and during 35 days after. After treatment with EFD, a slight, significant, curative effect was observed on days 13 and 15 with a faster recovery of weight loss (FIG. 31).

Clinical Scores

Scores were checked according to previous description (see Example I). The clinical symptoms decreased faster in EFD treated mice than in PBS treated mice (FIG. 32).

Inflammation of Colon

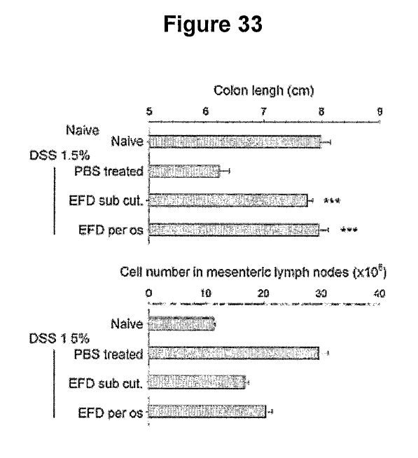

At the end of experiment (day 34), the colons of EFD treated mice were similar to those of naive mice never fed with DSS (FIG. 33 up).

Lymph Node and Spleen

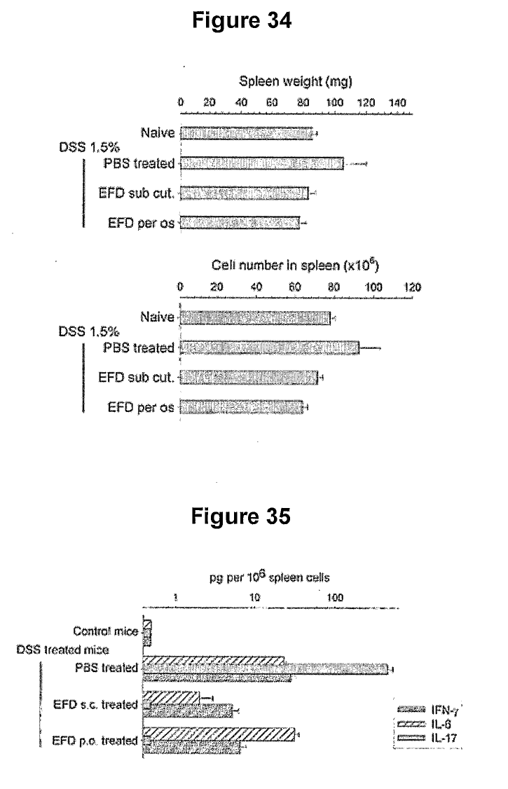

At the end of experiment (day 34), the number of cells in the lymph nodes of EFD treated mice were decreased compared to those of PBS treated mice, the difference being statistically different (FIG. 33 down). As illustrated in FIG. 34, the differences in spleen weight and number of spleen cells at day 34 are not statistically significant.

Inflammatory Regulators Release from Spleen

The spontaneous release of some inflammatory cytokines by spleen cells collected at the end of the experiment (day 34) was studied. The differences observed between PBS and EFD treatments were extremely significant for IL-17, not for IFN-γ and IL-6 (FIG. 35).

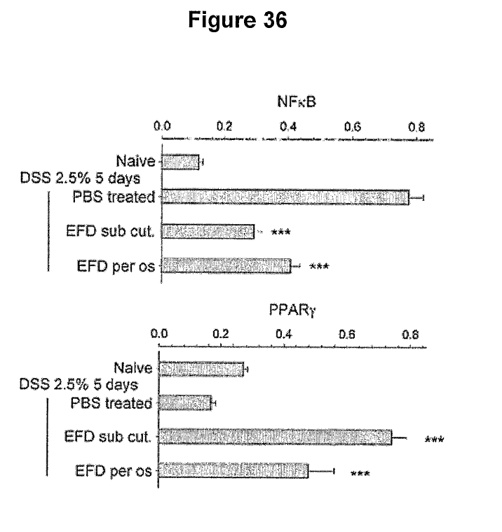

As illustrated in FIG. 36 up, a high concentration of NF-κB is maintained in spleens of PBS treated mice a month later after the end of DSS ingestion. At the same time, EFD treatment permits to have NF-κB concentrations significantly lower than without EFD treatment and similar to the ones of naive mice never fed with DSS.

At the end of experiment, levels of PPAR-γ observed in spleens of PBS and EFD treated mice are extremely different as illustrated by FIG. 36 down.

### Example IV

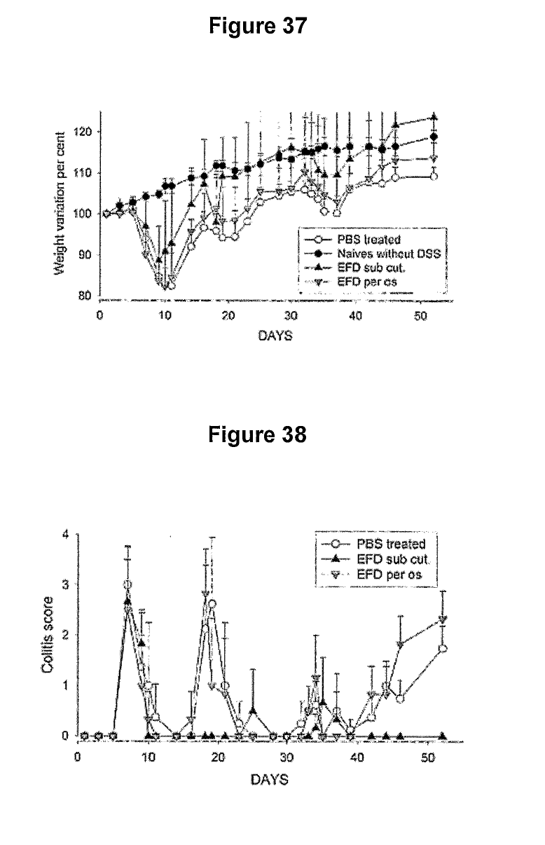

### Curative and Preventive Model of DSS Induced Chronic IBD (Long Term Effect) (FIGS. 37-42)

This model explored EFD curative and preventive treatments in chronic IBD.

### Protocol

C57BI/6 mice, 6 to 7 weeks old, were distributed in 4 groups of 10 mice. Mice (C57BI/6 male mice) received 1.5% DSS in their drinking water during 7 days, ordinary tap water during 8 days, 1.5% DSS in their drinking water during 5 days, tap water during 10 days, 1.5% DSS during 5 days and tap water thereafter. A group of mice were EFD treated: 100 μg given subcutaneously 9 days after the first DSS feeding; or 1 mg per os at days 9, 10 and 11 after the first DSS feeding. They did not receive more EFD treatment. The experiment ended at day 52.

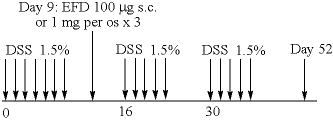

### Results

Weight Curves

In this model of curative/preventive treatment in chronic DSS-induced IBD, the weight of individual mouse was checked each day, 5 days a week from the first day of DSS ingestion and during 52 days after. After treatment with EFD, a slight, significant, curative effect was observed on days 13 and 15 with a faster and stable recovery of weight loss (FIG. 37).

Clinical Scores

Score were checked according to previous description (see Example I). The clinical symptoms decreased in subcutaneously EFD treated mice compared to in PBS treated mice (FIG. 38).

Inflammation of Colon

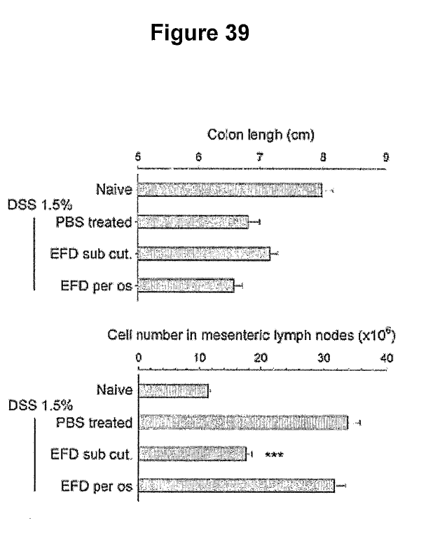

At the end of experiment (day 52), the colons of EFD treated mice were grossly similar to those of PBS treated mice (FIG. 39 up).

Lymph Node and Spleen

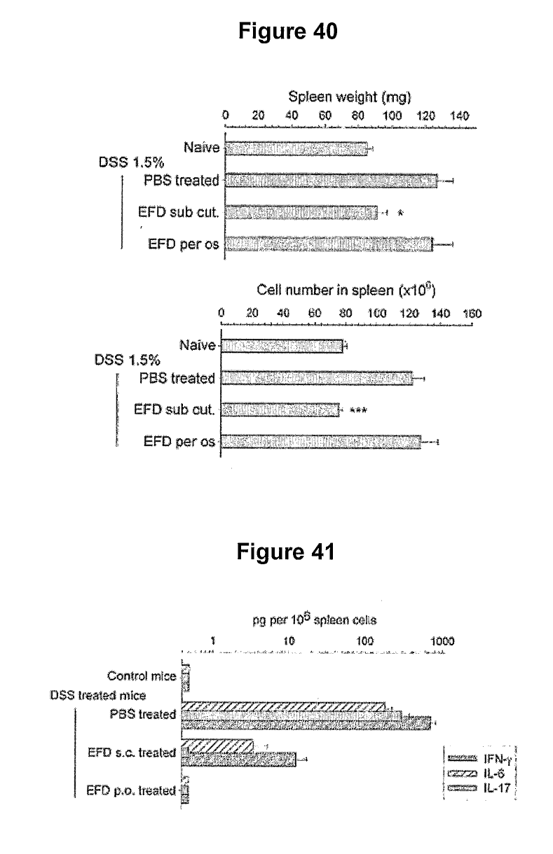

At the end of experiment (day 52), the number of cells were decreased only in the lymph nodes of subcutaneously EFD treated mice, the difference with PBS treated mice being extremely significant (FIG. 39 down). Similarly, spleen weight and spleen cell number were decreased only in mice which have been subcutaneously EFD treated (FIG. 40).

Inflammatory Regulators Release from Spleen

The spontaneous release of some inflammatory cytokines by spleen cells collected at the end of the experiment (day 52) was studied. The differences observed between PBS and EFD treatments were extremely significant, whatever the cytokine, but much marked for IL-17 (FIG. 41).

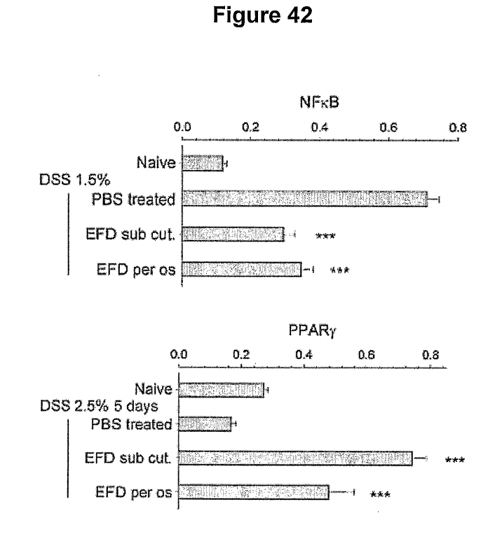

As illustrated in FIG. 42 up, after chronic IBD high concentration of NF-κB were observed in spleens of PBS treated mice. NF-κB concentrations in spleen of EFD treated mice were lower than the ones in spleen of PBS treated mice, the differences being extremely significant.

After chronic IBD, levels of PPAR-γ observed in spleens of PBS and EFD treated mice were very different, the differences being extremely significant (FIG. 42 down).

### Example V

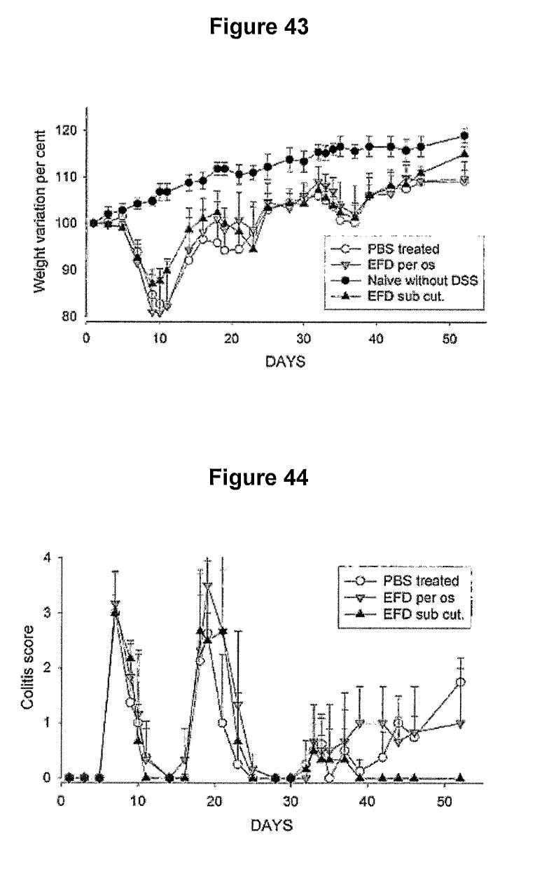

### Curative Model of DSS Induced Chronic IBD (Short Term Effect) (FIGS. 43-48)

This model explored EFD curative treatments in chronic IBD.

### Protocol

C57BI/6 mice, 6 to 7 weeks old, were distributed in 4 groups of 10 mice. Mice (C57BI/6 male mice) received 1.5% DSS in their drinking water during 7 days, ordinary tap water during 8 days, 1.5% DSS in their drinking water during 5 days, tap water during 10 days, 1.5% DSS during 5 days and tap water thereafter. A group of mice were EFD treated: 100 μg given subcutaneously 7 days after the last DSS feeding; or 1 mg per os at days 42, 43 and 45 after the first DSS feeding. The experiment ended at day 54.

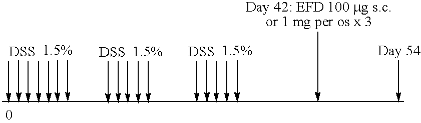

### Results

Weight Curves

In this model of curative treatment in chronic DSS-induced IBD, the weight of individual mouse was checked each day, 5 days a week from the first day of DSS ingestion and during 54 days after. After subcutaneous EFD treatment, a slight, curative effect was observed on day 54 with a recovery of weight loss (FIG. 43).

Clinical Scores

Score were checked according to previous description (see Example I). After only subcutaneous EFD treatment, the clinical symptoms decreased and get close to the normality (FIG. 44).

Inflammation of Colon

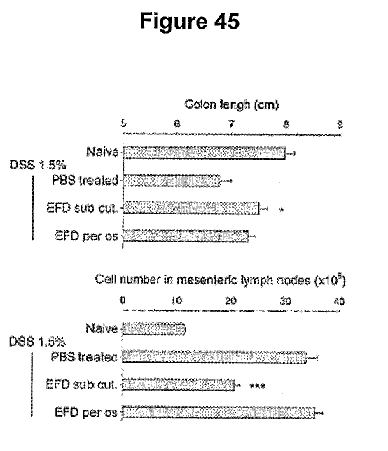

At the end of experiment (day 54), the colons of EFD treated mice were grossly similar to those of PBS treated mice (FIG. 45 up).

Lymph Node and Spleen

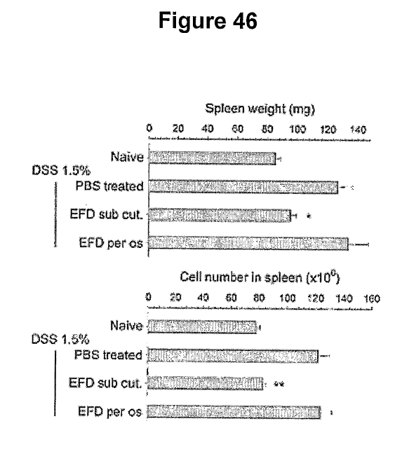

At the end of experiment (day 54), the number of cells were decreased only in the lymph nodes of subcutaneously EFD treated mice, the difference observed between EFD treated mice and PBS treated mice being extremely significant (FIG. 45 down). Similarly, spleen weight and spleen cell number were decreased only in mice which have been subcutaneously EFD treated (FIG. 46).

Inflammatory Regulators Release from Spleen

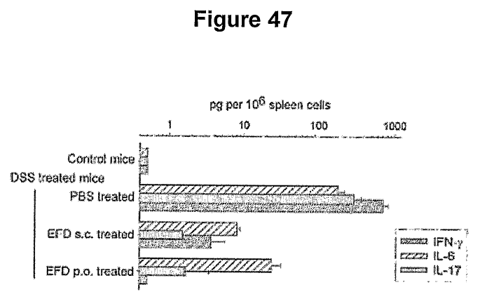

The spontaneous release of some inflammatory cytokines by spleen cells collected at the end of the experiment (day 54) was studied. The differences observed between PBS and EFD treatments were extremely significant, whatever the cytokine, but much marked for IL-17 (FIG. 47).

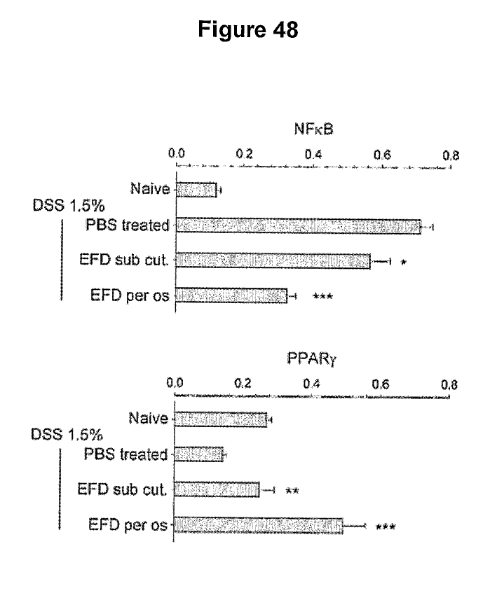

As illustrated in FIG. 48 up, after chronic IBD NF-κB concentrations in spleen of EFD treated mice were lower than the ones in spleen of PBS treated mice, the differences being very (subcutaneous treatment) or extremely (per os treatment) significant.

After chronic IBD, concentrations of PPAR-γ observed in spleens of PBS and EFD treated mice showed an extremely significant difference (FIG. 48 down). EFD treatment, whatever the mode of administration, permits to have a PPAR-γ concentration similar or lightly higher to the one of naive mice.

### Example VI

### Preventive Model of TNBS Induced IBD (Short Term Effect)

### Protocol

C57BI/6 male mice received or not TNBS (1 mg of 2,4,6-trinitrobenzene sulfonic acid (TNBS) in 100 μl of ethyl alcohol at 50% twice, locally in colon at day −5 and 0. Their stools were observed during 10 days. At day 10, the mice were weighted. A group of mice received EFD 100 μg subcutaneously 5 days before the first TNBS delivery (i.e. at day −10). A second group received PBS. The control group did not receive TNBS neither EFD.

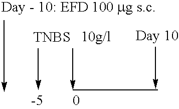

### Results

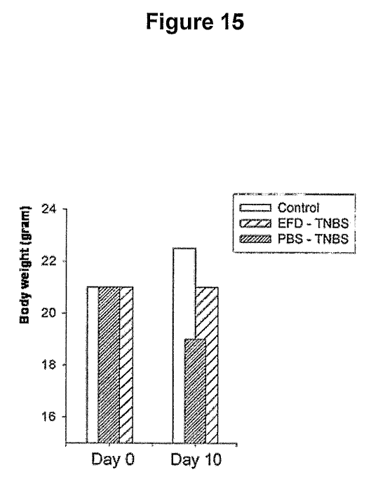

Weighing of the animals was made at day 0 and at day 10 after the beginning of TNBS treatment. Mice which have been received TNBS lost approximately 2 grams, when naive mice not treated with TNBS gained more than 1 gram and mice which have received EFD before TNBS had the same weight than at the beginning of experiment (FIG. 15).

### Conclusion of Examples

### DSS Models

Preventive models: EFD was given (subcutaneously or per os) 21 days before DSS feeding in an acute model (DSS feeding during 5 or 7 days—Example I) and in a chronic model (DSS feeding during 5 days, three times separated by 5 days—Example II). The protective effect was observed on clinical symptoms (body weight, aspect of stools), histopathological findings, cytokine/lymphokine concentrations in colonic tissues and spleen, levels of transcriptional factors NFκB and PPARγ in spleen.

Curative acute model: EFD was given (subcutaneously or per os) 24 hours after feeding with DSS during 5 days (Example III). In the EFD treated group the recovery of body weight was faster, the symptoms were less marked, the biological markers were closer to normal values than in the PBS treated group of mice at the end of experiment, one month later.

Curative and preventive chronic model: EFD was given (subcutaneously or per os) 48 hours after feeding with DSS during 7 days, 2 series of 5 days of DSS feeding were delivered before the end of experiment at day 52 (Example IV). In the EFD treated group the biological markers were closer to normal values than in the PBS treated group of mice, the effect on clinical findings was present but less marked.

Curative chronic model: EFD was given (subcutaneously or per os) 48 hours after a series of DSS feedings (DSS during 7 days and 2 series of 5 days of DSS feeding) (Example V). At the end of experiment at day 54, in the EFD treated group the biological markers were closer to normal values than in the PBS treated group of mice.

### TNBS Model

The experiment performed with TNBS included few mice with limited explorations. The decrease in inflammatory reactions observed after EFD treatment indicated that EFD is able to decrease pure T-lymphocyte dependent immune responses.

All these results indicate that EFD has potent anti-inflammatory activities in different DSS induced IBD models. As a preventive treatment, it acts on clinical symptoms and biological parameters. As a curative treatment, its acts essentially on biological parameters. The effects on clinical symptoms were present but less marked in these drastic models of chronic IBD, knowing that the delay between the treatment and the end of experiments (when measures are done) was short, 12 to 19 days.

The EFD efficacy was also observed in a TNBS induced IBD model (Example VI) demonstrating its anti-inflammatory potencies in pure T-lymphocyte dependent acquired immune responses.

In light of these examples, one skilled in the art may appreciate that the inventors have confirmed that the Gram positive bacteria preparation according to the present invention is useful for the prevention and treatment of intestinal inflammatory syndromes. Indeed, many cytokines (namely IFN gamma, IL-6 and IL-17) as well as two transcription factors (NFkappaB and PPARgamma) which are involved in the inflammatory response, have been assayed by the inventors. The results shown in the Example Section demonstrate that the Gram positive bacteria preparation according to the present invention has the original property of acting not only on the Th1 signalisation pathway to which TNF-alpha, IL-12, IFN-gamma and T-bet are associated with, but also on the Th2 signalisation pathway to which IL-4, IL-13 and GATA-3 are associated with, involving and on a new signalisation pathway to which IL-17 and PPAR-gamma seem to be associated with (Young Y. Current Gastroenterolo Rep 2006 December; 8 (6): 470-7).

1. The method of using a Gram positive bacteria preparation for the prevention and treatment of intestinal inflammatory syndromes, the preparation being characterized in that the Gram positive bacteria are killed or non infectious, and containing more than 50%, and preferentially more than 90%, of the bacterial protein components which are in a native structure.

2. The method of a Gram positive bacteria preparation as claimed in claim 1, wherein the killed or non infectious Gram positive bacteria preparation is obtained by using a process that does not denature the structures of the molecules contained in bacteria.

3. The method according to claim 1, characterized in that the Gram positive bacteria is Gram positive facultative intracellular bacteria.

4. The method according to claim 3, characterized in that the Gram positive facultative intracellular bacteria is Mycobacterium bovis BCG.

5. The method according to claim 1, characterized in that the Gram positive bacteria are killed by lyophilization.

6. The method according to claim 1, characterized in that the Gram positive bacteria are extended freeze-dried killed.

7. The method according to claim 1, characterized in that the intestinal inflammatory syndrome is an inflammatory bowel disease selected from the group consisting of Crohn's disease and ulcerative colitis.

8. A method for preventing or treating a disease caused by a Th1/Th2 imbalance, the method comprising the steps of:
a) providing a killed or non infectious Gram positive bacteria preparation or a portion thereof retaining the capacity to inhibit intestinal inflammatory syndromes, and
b) administering an effective amount of the killed Gram positive bacteria preparation or the portion thereof to a patient affected by the disease.

9. The method according to claim 8, characterized in that the disease is an inflammatory bowel disease selected from the group consisting of Crohn's disease and ulcerative colitis.

10. The method according to claim 8, characterized in that the Gram positive bacteria is Gram positive facultative intracellular bacteria.

11. The method according to claim 10, characterized in that the Gram positive facultative intracellular bacteria is Mycobacterium bovis BCG.

12. A method for preventing or treating an intestinal inflammatory syndrome, the method comprising the step of administering to a patient an effective amount of a killed Gram positive bacteria preparation, thereby stimulating the production of leukocytic regulatory cells.

13. The method according to claim 12, characterized in that the intestinal inflammatory syndrome is Crohn's disease or ulcerative colitis.

14. The method according to claim 12, characterized in that the Gram positive bacteria cell is killed by lyophilization.

15. The method according to claim 12, characterized in that the Gram positive bacteria cell is extended freeze-dried killed.

16. The method according to claim 12, characterized in that the Gram positive bacteria cell is a Gram positive facultative intracellular bacteria.

17. The method according to claim 16, characterized in that the Gram positive facultative intracellular bacteria is Mycobacterium bovis BCG.

18. The method according to claim 12, characterized in that the leukocytic regulatory cells are CD4+, CD25+, T cells, B cells and/or dendritic cells.

19. A composition for the treatment and/or prevention of intestinal inflammatory bowel diseases containing a Gram positive bacteria composition prepared by the following steps:
a. harvesting a culture of live bacteria cells,
b. washing the bacteria cells in water or in an aqueous solution of a salt such as borate,
c. freezing the bacteria cells in water or in an aqueous solution of a salt such as borate,
d. killing the frozen bacteria cells by drying them in a freeze-dryer, for a time sufficient to remove at least 98.5% of the water, and
e. collecting the extended freeze-dried bacteria cells.

20. The method of using a Gram positive bacteria preparation for the manufacture of a medicament for preventing or treating intestinal inflammatory bowel diseases, wherein, in said Gram positive bacteria preparation, the Gram positive bacteria are killed or non infectious, and containing more than 50%, of the bacterial protein components which are in a native structure.

21. The method according to claim 20, wherein the killed or non infectious Gram positive bacteria preparation is obtained by using a process that does not denature the structures of the molecules contained in bacteria.

22. The method according to claim 20, wherein Gram positive bacteria preparation is prepared by the following steps:
a. harvesting a culture of live bacteria cells,
b. washing the bacteria cells in water or in an aqueous solution of a salt such as borate,
c. freezing the bacteria cells in water or in an aqueous solution of a salt such as borate,
d. killing the frozen bacteria cells by drying them in a freeze-dryer, for a time sufficient to remove at least 98.5% of the water, and
e. collecting the extended freeze-dried bacteria cells.

23. The method according to claim 20, characterized in that the Gram positive bacteria is Gram positive facultative intracellular bacteria.

24. the method according to claim 23, characterized in that the Gram positive facultative intracellular bacteria is Mycobacterium bovis BCG.

25. The method according to claim 20, characterized in that the Gram positive bacteria are killed by lyophilization.

26. The method according to claim 20, characterized in that the Gram positive bacteria are extended freeze-dried killed.

27. The method according to claim 20, characterized in that the inflammatory bowel disease is selected from the group consisting of Crohn's disease and ulcerative colitis.

28. The method of preparing a Gram positive bacteria preparation for the prevention and treatment of a disease caused by a Th1/Th2 imbalance, the preparation being characterized in that the Gram positive bacteria are killed or non infectious, and containing more than 50%, and preferentially more than 90%, of the bacterial protein components which are in a native structure.

29. The method according to claim 28, characterized in that the disease is an inflammatory bowel disease selected from the group consisting of Crohn's disease and ulcerative colitis.

30. The method according to claim 28, characterized in that the Gram positive bacteria is Gram positive facultative intracellular bacteria.

31. Use the method according to claim 30, characterized in that the Gram positive facultative intracellular bacteria is Mycobacterium bovis BCG.

32. The method of using of a Gram positive bacteria preparation for the manufacture of a medicament for preventing or treating a disease caused by a Th1/Th2 imbalance, the preparation being characterized in that the Gram positive bacteria are killed or non infectious, and containing more than 50%, and preferentially more than 90%, of the bacterial protein components which are in a native structure.

33. The method according to claim 32, characterized in that the disease is an inflammatory bowel disease selected from the group consisting of Crohn's disease and ulcerative colitis.

34. The method according to claim 32, characterized in that the Gram positive bacteria is Gram positive facultative intracellular bacteria.

35. The method according to claim 34, characterized in that the Gram positive facultative intracellular bacteria is Mycobacterium bovis BCG.
